(19) United States
(12) Patent Application Publication
(10) Pub. No.: US 2016/0115558 A1
(43) Pub. Date: Apr. 28, 2016

(54) CONTROL OF METABOLIC FLUX IN CELL-FREE BIOSYNTHETIC SYSTEMS

(71) Applicants: GREENLIGHT BIOSCIENCES, INC., Medford, MA (US); THE BOARD OF TRUSTEES OF THE LELAND STANFORD JUNIOR UNIVERSITY, Palo Alto, CA (US)

(72) Inventor: James R. Swartz, Menlo Park, CA (US)

(73) Assignees: GreenLight Biosciences, Inc., Medford, MA (US); The Board of Trustees of the Leland Stanford Junior University, Stanford, CA (US)

(21) Appl. No.: 14/895,992

(22) PCT Filed: Jun. 5, 2014
(86) PCT No.: PCT/US14/41009

§ 371 Date: Dec. 4, 2015

Related U.S. Application Data
(60) Provisional application No. 61/831,376, filed on Jun. 5, 2013.

(51) Int. Cl. C12Q 3/00

(52) U.S. Cl. CPC C12Q 3/00

(57) ABSTRACT

Methods are provided for controlling metabolic flux rate in a cell-free system comprising a complex set of enzymes, to produce a desired product of a pathway of interest. In the methods of the invention, measurements of metabolic performance parameters are taken by continuous monitoring or intermittent monitoring. Based on the metabolic performance parameters, the system is modified by one or more steps comprising: (i) altering enzyme levels in the cell-free system; (ii) altering feed rate of a substrate that controls redox flux or carbon flux to the cell-free system; (iii) altering O₂ addition to the cell-free system; (iv) controlling efficiency of electron transport system by altering leakage across a membrane; wherein enzymes present in the pathway of interest catalyze production of a desired product.

### RELATED APPLICATIONS

The present invention claims priority under 35 U.S.C. §119(e) to U.S. provisional patent application, U.S. Ser. No. 61/831,376, filed Jun. 5, 2013, which is incorporated herein by reference.

### BACKGROUND OF THE INVENTION

Production of chemicals via synthetic enzymatic pathways in microbial hosts has proven useful for many important classes of molecules, including isoprenoids, polyketides, nonribosomal peptides, bioplastics, and chemical building blocks. Due to the inherent modularity of biological information, synthetic biology holds great potential for expanding this list of microbially produced compounds even further. Yet embedding a novel biochemical pathway in the metabolic network of a host cell can disrupt the subtle regulatory mechanisms that the cell has evolved over the millennia. Indeed, the final yield of a compound is often limited by deleterious effects on the engineered cell's metabolism that are difficult to predict due to limited understanding of the complex interactions that occur within the cell. The unregulated consumption of cellular resources, metabolic burden of heterologous protein production, and accumulation of pathway intermediates/products that are inhibitory or toxic to the host are all significant issues that may limit overall yield.

The concept of metabolic engineering has emerged to fulfill this purpose, which can be defined as purposeful modification of metabolic and cellular networks by employing various experimental techniques to achieve desired goals. What distinguishes metabolic engineering from genetic engineering and strain improvement is that it considers metabolic and other cellular network as a whole to identify targets to be engineered. In this sense, metabolic flux is an essential concept in the practice of metabolic engineering. Although gene expression levels and the concentrations of proteins and metabolites in the cell can provide clues to the status of the metabolic network, they have inherent limitations in fully describing the cellular phenotype due to the lack of information on the correlations among these cellular components. Metabolic fluxes represent the reaction rates in metabolic pathways, and serve to integrate these factors through a mathematical framework. Thus, metabolic fluxes can be considered as one way of representing the phenotype of the cell as a result of interplays among various cell components; the observed metabolic flux profiles reflect the consequences of interconnected transcription, translation, and enzyme reactions incorporating complex regulations.

Cell-free synthesis may offer advantages over in vivo production methods. Cell-free systems can direct most, if not all, of the metabolic resources of the cell towards the exclusive production from one pathway. Moreover, the lack of a cell wall in vitro is advantageous since it allows for control of the synthesis environment.

As the environments of most organisms are constantly changing, the reactions of metabolism are finely regulated to maintain homeostatic conditions within cells. Metabolic pathways are controlled by regulating the activity of enzymes within a pathway, by altering the activity of the protein, e.g. through allosteric inhibition and the like; and by altering the expression or translation of the enzyme as well as its stability; i.e., its useful lifetime. Pathways are also regulated by altering the concentration of substrates and cofactors that are present in the cell.

Among the molecules that affect flux through a pathway are the coenzymes, including ATP. This nucleotide is used to transfer chemical energy between different chemical reactions, and serves as a carrier of phosphate groups in phosphorylation reactions. Nicotinamide adenine dinucleotide (NADH), a derivative of vitamin B3 (niacin), is an important coenzyme that acts as an electron carrier. It exists in two related forms in the cell, NADH and NADPH. Many separate types of dehydrogenases remove electrons from their substrates and reduce NAD+ into NADH. This reduced form of the coenzyme is then a substrate for any of the reductases in the cell that need to reduce their substrates.

Many enzymatic reactions are oxidation-reduction reactions in which one compound is oxidized and another compound is reduced. The ability of an organism to carry out oxidation-reduction reactions depends on the oxidation-reduction (redox) state of the environment, or its reduction potential. While this is sometimes expressed by a single metric, a more useful analysis will examine the redox state of important redox reagents, in particular, the NAD+ and NADP+ coenzymes. The presence and activity of particular redox (or electron transfer) enzymes will then determine the relative redox state of different redox reagents. For example, the enzyme glutathione reductase catalyzes the transfer of electrons from NADPH to oxidized glutathione to form reduced glutathione and NADP+. Depending upon the rate of that reaction and other factors, the redox state of the NADPH/NAD+ pair may or may not be approximately equivalent to the redox state of the oxidized/reduced glutathione pair. While living cells have developed many strategies to closely regulate the intracellular redox states of different such redox pairs, through regulation of pathways and redox buffers, e.g. glutathione and/or ascorbate, cell-free systems may require engineering to provide for such regulation and are particularly suitable for such engineering and control.

It is desirable to manipulate parameters that influence the metabolic flux rates of key metabolites and pathways during cell-free biosynthetic reactions in order to optimize conditions that influence system performance, which parameters may reflect a balance between utilization of a carbon source, such as glucose, through different pathways and may also optimize ATP production in relation to NADH, and NADPH production. The present invention addresses this issue.

### SUMMARY OF THE INVENTION

Compositions and methods are provided for monitoring and controlling metabolic flux rates in a cell-free system comprising a complex set of enzymes, during biosynthesis of a desired product of a pathway of interest. The methods of the invention monitor key metabolic parameters of central metabolism, which parameters may include, without limitation, concentrations of NADP(H); NAD(H); ATP; ribulose-5-phosphate; consumption of a carbon source, such as glucose; and O₂ consumption. For the pathway of interest, desired target levels of one or more of the metabolic parameters are determined, for example through empirical screening methods, or deduction from known metabolic pathway equations. By monitoring the cell-free system for these key metabolic parameters during biosynthesis, and determining the deviation from desired levels, information is obtained regarding the metabolic state of the system. Adjusting metabolic performance based on the measurements is performed by one or more steps comprising: (i) altering enzyme levels in the cell-free system; (ii) altering feed rate of a substrate that controls redox flux or carbon flux to the cell-free system; (iii) altering O₂ addition to the cell-free system; (iv) controlling efficiency of electron transport system by altering leakage across a membrane; wherein enzymes present in the pathway of interest catalyze production of the desired product.

Some of the key metabolic parameters of interest for the methods of the invention relate to central metabolism, including the pathways for glycolysis and pentose shunt; oxidative phosphorylation; and the redox flux, e.g. between NAD, NADH, NADP and NADPH, for example as diagrammed in FIG. 1.

Various methods may be employed to alter the metabolic flux rate. In some embodiments, exogenous enzymes involved in redox flux pathways are provided to the reaction mixture as required in order to achieve the desired redox balance, either in the form of protein or in the form of a coding sequence for the protein. In other embodiments, the microbial cell utilized in the initial reaction mixture is genetically modified to alter the expression and/or composition of enzymes involved in redox flux pathways in order to provide an optimized initial condition for the reactions. In other embodiments, targeted enzymes are engineered to comprise a unique recognition sequence for proteolytic cleavage, so that enzyme activity can readily be reduced if necessary. As these enzymes are involved in central metabolism, it may be necessary to modulate expression in a manner that does not affect the growing cells, e.g. by relocation or secretion of the enzyme.

In the methods of the invention, a microbial cell, which may be genetically modified or may be a wild-type cell, is grown to a desired density, then lysed and the lysate, which may be a crude lysate, is combined with substrate(s) and an energy source if needed during a production phase, and incubated for a period of time sufficient to generate desired product of a pathway of interest. Additional substrate, nutrients, cofactors, buffers, reducing agents, and/or ATP generating systems, may be added to the cell-free system. Genetic modifications of interest to the microbial cell include the introduction of heterologous enzymes to provide for non-native enzymatic activities, and may further include deletion or down-regulation of undesirable enzyme activity; as well as enhancement or upregulation of native enzymes. During the production phase, at least one and preferably two or more key metabolic parameters are monitored, where the monitoring may be continuous or intermittent. Based on the targeted levels of key metabolic parameters, which may be pre-determined target levels, the metabolic performance is adjusted as described above.

In some embodiments, methods are provided for producing a product of interest at a high flux rate, the method comprising: growing cells; lysing the cells; and producing the product of the pathway in a cell-free system comprising the lysate, where metabolic flux rates of key parameters are monitored and controlled.

In some embodiments, a metabolic parameter for monitoring and adjusting is the concentration of a nicotinamide adenine dinucleotide, for example one or more of NAD, NADH, NADP and NADPH. In some embodiments, a metabolic parameter for monitoring and adjusting is the concentration of ATP or ADP. In some embodiments, a metabolic parameter for monitoring and adjusting is the dissolved O₂ concentration.

In some embodiments, activity of one or more enzymes selected from glucose-6 phosphate dehydrogenase, glucose phosphate isomerase, and transhydrogenase is adjusted in response to metabolic parameter monitoring.

In some embodiments a compound that increases leakage of electrons in added to the cell-free system, e.g. a protonophore may be added, such as 2,4-dinitrophenol (DNP); Carbonyl cyanide-p-trifluoromethoxyphenylhydrazone (FCCP); and/or Carbonyl cyanide m-chlorophenyl hydrazone (CCCP).

### BRIEF DESCRIPTION OF THE DRAWINGS

FIG. 1 is a schematic of certain aspects of central metabolism.

FIG. 2 is diagram of a sample metabolically engineered network for the production of homoserine from aspartate, e.g., using a metabolic control test rig. The production pathway consists of three enzymatic steps requiring two NADPH molecules and one ATP molecule per product molecule. The diagram also indicates potential parameters for monitoring and control.

FIG. 3 is a schematic of a metabolic control test rig.

### DETAILED DESCRIPTION OF CERTAIN EMBODIMENTS OF THE INVENTION

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are incorporated herein by reference, the definition set forth in this section prevails over the definition that is incorporated herein by reference.

Citation of publications or documents is not intended as an admission that any of such publications or documents are pertinent prior art, nor does it constitute any admission as to the contents or date of these publications or documents.

As used herein, “a” or “an” means “at least one” or “one or more” unless otherwise indicated.

Nucleic Acids.

The nucleic acids used to practice this invention, whether RNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; and U.S. Pat. No. 4,458,066, each incorporated herein by reference.

Host cells of interest for pathway engineering include a wide variety of heterotrophic and autotrophic microorganisms, including bacteria, fungi and protozoans. Species of interest include, without limitation, S. cerevisiae, E. coli, B. subtilis, and Picchia pastoris.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993), each incorporated herein by reference.

Flux.

The term “flux” as used herein refers to the rate that molecules pass through a pathway or reaction of interest. Among the factors that control flux are rate of catalysis of enzymes in the pathway, the availability of substrate, the concentration of enzymes in a cell, and/or the proximity of enzymes in a pathway.

While a high rate of flux through a pathway of interest is desirable, at the same time it can create toxicity issues if a product not normally accumulated at high levels in the cell is produced at a high rate. A stressed cell produces a number of proteins undesirable for maintaining active biocatalysis, such as nucleases, heat shock proteins, proteases and the like.

The methods of the invention provide a means of controlling flux through a pathway or pathways in a cell-free extract such that the desired product or products are preferentially produced.

Methods of determining flux rates are known and used in the art, for example as described by Wiechert et al. (2001) Metab. Eng. 3, 265-283 and Metab Eng. 2001 July; 3(3):195-206; or metabolic engineering texts such as Lee and Papoutsakis, 1999, Stephanopoulos, Aristidou, Nielsen, 1998, Nielsen and Eggeling, 2001, each incorporated herein by reference. Flux may be calculated from measurable quantities using techniques such as metabolic flux analysis (MFA), for example by direct measurement of the conversion of isotopically labeled substrate or by simultaneously measuring the rates of glucose consumption, oxygen consumption, and CO₂ production. Using the methods of this invention, flux rates may also be measured directly, for example, by measuring the rate of increase in product concentration or by measuring the intensity of light production from an ATP dependent luciferase.

Metabolic Parameters.

Parameters are quantifiable components or properties of the cell-free system, particularly those that can be accurately measured, desirably in a high throughput system. For the purposes of the present invention, parameters of interest are usually parameters associated with central metabolism, including without limitation nucleotides, e.g. ATP, GTP; carbon and energy sources, such as glucose, pyruvate; nicotinamide adenine dinucleotides, e.g. NAD, NADH, NADP, NADPH; O₂ consumption rate and dissolved oxygen concentration; pH; and the like. Parameters of interest can be monitored continuously or intermittently, e.g. with a pH meter; real time HPLC analysis, real-time enzyme assays, by measuring the gas concentration in the exit gas stream and conducting a material balance; a rapid turnaround HPLC/MS instrument. Rate of ATP production may be determined by taking a side stream of the reactor contents into a flow cell where luciferase and luciferin are added and the resultant luminescence intensity measured.

While most parameters will provide a quantitative readout, in some instances a semi-quantitative or qualitative result will be acceptable. Readouts may include a single determined value, or may include mean, median value or the variance. Markers are selected to serve as parameters based on the following criteria, where any parameter need not have all of the criteria: the parameter is modulated in the biosynthetic reaction; the parameter is modulated by a factor, e.g. an enzyme, substrates, that is available; the parameter has a robust response that can be easily detected and differentiated. The set of parameters is selected to allow monitoring of the central metabolism processes of interest.

Pre-Determination of Target Parameter Levels.

For the pathway of interest, desired target levels of one or more of the metabolic parameters are determined, for example through empirical screening methods, or deduction from known metabolic pathway equations. By monitoring the cell-free system for these key metabolic parameters during biosynthesis, and determining the deviation from desired levels, information is obtained regarding the metabolic state of the system.

Empirical analysis may be performed by conducting biosynthesis of the product of interest, and measuring the yield while monitoring the target parameter. The yield may be further measured in the presence of one or more agents or adjustments to the system, in order to determine the effect on overall biosynthesis. For example, agents such as protonophores, enzymes, and/or O₂, are added to at least one reaction condition and usually a plurality of conditions, often while comparing to a control reaction lacking the agent. The change in parameter readout in response to the agent is measured, desirably normalized, and evaluated by comparison to other reaction conditions.

The agents are conveniently added in solution, or readily soluble form, to the cell-free system. The agents may be added in a flow-through system, as a stream, intermittent or continuous, or alternatively, adding a bolus of the compound, singly or incrementally, to an otherwise static solution. Preferred agent formulations do not include additional components, such as preservatives, that may have a significant effect on the overall formulation.

The data may be input to a data processing system, and may be automated for analysis of the parameters. The data processing unit may further be connected to an automated system for introduction of parameter modulating agents, e.g. enzymes, O₂, and/or protonophores.

Yield.

The term “yield” as used herein refers to the final volumetric concentration of product molecules that can be accumulated during the course of a batch or fed-batch reaction, or can refer to the product concentration that can be maintained during continuous operation.

Transhydrogenase.

The energy-transducing nicotinamide nucleotide transhydrogenase is an enzyme that catalyzes the direct transfer of a hydride ion between NAD(H) and NADP(H) in a reaction that is coupled to transmembrane proton translocation. The proton motive force accelerates the rate of hydride ion transfer from NADH to NADP+, and shifts the equilibrium of this reaction toward NADPH formation. Transhydrogenation in the reverse direction from NADPH to NAD is accompanied by outward proton translocation and formation of a proton motive force. In reverse transhydrogenation, the enzyme utilizes substrate binding energy for proton pumping. In addition, soluble pyridine nucleotide transhydrogenases are not membrane associated and primarily function to reoxidize NADPH to NADP+ while reducing NAD+ to NADH.

Glucose-6-phosphate Dehydrogenase

(G6PD or G6PDH) converts glucose-6-phosphate into 6-phosphoglucono-δ-lactone and is the rate-limiting enzyme of the pentose phosphate pathway. EC number 5.3.1.9; CAS number 9001-41-6.

Glucose-6-phosphate Isomerase,

(alternatively known as phosphoglucose isomerase or phosphohexose isomerase), is an enzyme that catalyzes the conversion of glucose-6-phosphate into fructose 6-phosphate in the second step of glycolysis.

Enzyme Pathway.

As used herein, the term “enzyme pathway” or “pathway of interest” refers to a cellular system for converting a substrate to a product of interest, where the system comprises a plurality of enzymes and may additionally comprise substrates acted upon by one or more of the enzymes, products of the enzyme-catalyzed reaction, co-factors utilized by the enzymes, and the like. For the purposes of the present invention, the pathway is present in a lysate of a cell. Many metabolic pathways are known and have been described in microbial systems, and are accessible in public databases. For example, a number of reference books are available, including, inter alia, The Metabolic Pathway Engineering Handbook (2009), ed. C. Smolke, CRC, ISBN-10: 1420077651 and 1439802963; Metabolic Engineering: Principles and Methodologies (1998) Stephanopoulos, Academic Press ISBN-10: 0126662606, Greenberg D M. Metabolic Pathways: Energetics, tricarboxylic acid cycle, and carbohydrates. Academic Press; 1967; Greenberg M. Metabolic pathways. Academic Press; 1968; Greenberg DM. Metabolic pathways. Academic; 1970; and Greenberg D M, Vogel H J. Metabolic pathways. Academic; 1971, each incorporated herein by reference.

Pathways of interest include, without limitation, pathways involved in carbohydrate, amino acid, nucleic acid, steroid, and fatty acid metabolism, and may include synthesis of antibiotics, e.g. actinomycin, bleomycin, rifamycin, chloramphenicol, tetracycline, lincomycin, erythromycin, streptomycin, cyclohexamide, puromycin, cycloserine, bacitracin, penicillin, cephalosporin, vancomycin, polymyxin, and gramicidin; biosurfactants e.g. rhamnolipids, sophorolipids, glycolipids, and lipopeptides; biological fuels e.g. bioethanol, biodiesel, and biobutanol; amino acids e.g. L-glutamate, L-lysine, L-phenylalanine, L-aspartic acid, L-isoleucine, L-valine, L-tryptophan, L-proline (hydroxyproline), L-threonine, L-methionine, and D-p-hydroxyphenylglycine; organic acids e.g. citric acid, lactic acid, gluconic acid, acetic acid, propionic acid, succinic acid, fumaric acid, and itaconic acid; fatty acids e.g. arachidonic acid, polyunsaturated fatty acid (PUBA), and γ-linoleic acid; polyols e.g. glycerol, mannitol, erythritol, and xylitol; flavors and fragrances e.g. vanillin, benzaldehyde, dixydroxyacetone, 4-(R)-decanolide, and 2-actyl-1-pyrroline; nucleotides e.g. 5′-guanylic acid and 5′-inosinic acid; vitamins e.g. vitamin C, vitamin F, vitamin B2, provitamin D2, vitamin B12, folic acid, nicotinamide, biotin, 2-keto-L-gulonic acid, and provitamin Q10; pigments e.g. astaxathin, β-carotene, leucopene, monascorubrin, and rubropunctatin; sugars and polysaccharides e.g. ribose, sorbose, xanthan, gellan, and dextran; biopolymers and plastics e.g. polyhydroxyalkanoates (PHA), poly-γ-glutamic acid, and 1,3-propanediol; and the like as known in the art.

A number of reactions may be catalyzed by enzymes in pathways of interest. Broad classes, which can be identified by enzyme classification number, provided in parentheses, include (EC 1) oxidoreductases, e.g. dehydrogenases, oxidases, reductases, oxidoreductases, synthases, oxygenases, monooxygenases, dioxygenases, lipoxygenases, hydrogenases, transhydrogenases, peroxidases, catalases, epoxidases, hydroxylases, demethylases, desaturases, dismutases, hydroxyltransferases, dehalogenases, deiodinases; (EC2) transferases, e.g. Transaminases, kinases, dikinases, methyltransferases, hydroxymethyltransferases, formyltransferases, formiminotransferases, carboxytransferases, carbamoyltransferases, amidinotransferases, transaldolases, transketolases, acetyltransferases, acyltransferases palmitoyltransferases, succinyltransferases, malonyltransferases, galloyltransferases, sinapoyltransferases, tigloyltransferases, tetradecanoyltransferases, hydroxycinnamoyltransferases, feruloyltransferases, mycolyltransferases, benzoyltransferases, piperoyltransferases, trimethyltridecanoyltransferase, myristoyltransferases, coumaroyltransferases, thiolases, aminoacyltransferases, phosphorylases, hexosyltransferases, pentosyltransferases, sialyltransferases, pyridinylases, diphosphorylases, cyclotransferases, sulfurylases, adenosyltransferases, carboxyvinyltransferases, isopentenyltransferases, aminocarboxypropyltransferases, dimethylallyltransferases, farnesyltranstransferases, hexaprenyltranstransferases, decaprenylcistransferases, pentaprenyltranstransferases, nonaprenyltransferases, geranylgeranyltransferases, aminocarboxypropyltransferases, oximinotransferases, purinetransferases, phosphodismutases, phosphotransferases, nucleotidyltransferases, polymerases, cholinephosphotransferases, phosphorylmutases, sulfurtransferases, sulfotransferases, CoA-transferases; (EC3) hydrolases, e.g. lipases, esterases, amylases, peptidases, hydrolases, lactonases, deacylases, deacetylases, pheophorbidases, depolymerases, thiolesterases, phosphatases, diphosphatases, triphosphatases, nucleotidases, phytases, phosphodiesterases, phospholipases, sulfatases, cyclases, oligonucleotidases, ribonucleases, exonucleases, endonucleases, glycosidases, nucleosidases, glycosylases, aminopeptidases, dipeptidases, carboxypeptidases, metallocarboxypeptidases, omega-peptidases, serine endopeptidases, cystein endopeptidases, aspartic endopeptidases, metalloendopeptidases, threonine endopeptidases, aminases, amidases, desuccinylases, deformylases, acylases, deiminases, deaminases, dihydrolases, cyclohydrolases, nitrilases, ATPases, GTPases, halidases, dehalogenases, sulfohydrolases; (EC 4) lyases, e.g. decarboxylases, carboxylases, carboxykinases, aldolases, epoxylyases, oxoacid-lyases, carbon-carbon lyases, dehydratases, hydratases, synthases, endolyases, exolyases, ammonia-lyases, amidine-lyases, amine-lyases, carbon-sulfur lyases, carbon-halide lyases, phosphorus-oxygen lyases, dehydrochlorinases; (EC 5) isomerases, e.g. isomerases, racemases, mutases, tautomerases, phosphomutases, phosphoglucomutases, aminomutases, cycloisomerase, cyclases, topoisomerases; and (EC 6) ligases, e.g. synthetases, tNRA-ligases, acid-thiol ligases, amide synthases, peptide synthases, cycloligases, carboxylases, DNA-ligases, RNA-ligases, cyclases.

More specific classes include, without limitation oxidoreductases, including those (EC 1.1) acting on the CH—OH group of donors, and an acceptor; (EC 1.2) Acting on the aldehyde or oxo group of donors, and an acceptor; (EC 1.3) Acting on the CH—CH group of donors, and an acceptor; (EC 1.4) Acting on the CH—NH2 group of donors, and an acceptor; (EC 1.5) Acting on the CH—NH group of donors, and an acceptor; (EC 1.6) Acting on NADH or NADPH, and an acceptor; (EC 1.7) Acting on other nitrogenous compounds as donors, and an acceptor; (EC 1.8) Acting on a sulfur group of donors, and an acceptor; (EC 1.9) Acting on a heme group of donors, and an acceptor; (EC 1.1) Acting on diphenols and related substances as donors, and an acceptor; (EC 1.11) Acting on a peroxide as acceptor; (EC 1.12) Acting on hydrogen as donor, and an acceptor; (EC 1.13) Acting on single donors with incorporation of molecular oxygen, incorporating one or two oxygen atoms; (EC 1.14) Acting on paired donors, with incorporation or reduction of molecular oxygen, with the donor being 2-oxoglutarate, NADH, NADPH, reduced flavin, flavoprotein, pteridine, iron-sulfur protein, ascorbate; (EC 1.15) Acting on superoxide radicals as acceptor; (EC 1.16) Oxidising metal ions, and an acceptor; (EC 1.17) Acting on CH or CH2 groups, and an acceptor; (EC 1.18) Acting on iron-sulfur proteins as donors, and an acceptor; (EC 1.19) Acting on reduced flavodoxin as donor, and an acceptor; (EC 1.2) Acting on phosphorus or arsenic in donors, and an acceptor; (EC 1.21) Acting on X—H and Y—H to form an X—Y bond, and an acceptor; where acceptors for each donor category may include, without limitation: NAD, NADP, heme protein, oxygen, disulfide, quinone, an iron-sulfur protein, a flavin, a nitrogenous group, a cytochrome, dinitrogen, and H⁺.

Transferases include those: (EC 2.1) Transferring one-carbon groups; (EC 2.2) Transferring aldehyde or ketonic groups; (EC 2.3) Acyltransferases; (EC 2.4) Glycosyltransferases; (EC 2.5) Transferring alkyl or aryl groups, other than methyl groups; (EC 2.6) Transferring nitrogenous groups; (EC 2.7) Transferring phosphorus-containing groups; (EC 2.8) Transferring sulfur-containing groups; (EC 2.9) Transferring selenium-containing groups.

Hydrolases include those: (EC 3.1) Acting on ester bonds; (EC 3.2) Glycosylases; (EC 3.3) Acting on ether bonds; (EC 3.4) Acting on peptide bonds (peptidases); (EC 3.5) Acting on carbon-nitrogen bonds, other than peptide bonds; (EC 3.6) Acting on acid anhydrides; (EC 3.7) Acting on carbon-carbon bonds; (EC 3.8) Acting on halide bonds; (EC 3.9) Acting on phosphorus-nitrogen bonds; (EC 3.1) Acting on sulfur-nitrogen bonds; (EC 3.11) Acting on carbon-phosphorus bonds; (EC 3.12) Acting on sulfur-sulfur bonds; (EC 3.13) Acting on carbon-sulfur bonds.

Lyases include those: (EC 4.1) Carbon-carbon lyases; (EC 4.2) Carbon-oxygen lyases; (EC 4.3) Carbon-nitrogen lyases; (EC 4.4) Carbon-sulfur lyases; (EC 4.5) Carbon-halide lyases; (EC 4.6) Phosphorus-oxygen lyases.

Isomerases include those: (EC 5.1) Racemases and epimerases; (EC 5.2) cis-trans-Isomerases; (EC 5.3) Intramolecular isomerases; (EC 5.4) Intramolecular transferases (mutases); (EC 5.5) Intramolecular lyases.

Ligases, include those: (EC 6.1) Forming carbon-oxygen bonds; (EC 6.2) Forming carbon-sulfur bonds; (EC 6.3) Forming carbon-nitrogen bonds; (EC 6.4) Forming carbon-carbon bonds; (EC 6.5) Forming phosphoric ester bonds; (EC 6.6) Forming nitrogen-metal bonds.

Enzymes in a pathway may be naturally occurring, or modified to optimize a characteristic of interest, e.g. substrate specificity, reaction kinetics, solubility, and/or insensitivity to feedback inhibition. In addition, in some cases, the gene expressing the enzyme will be optimized for codon usage. In some embodiments the complete pathway comprises enzymes from a single organism, however such is not required, and combining enzymes from multiple organisms is contemplated. For some purposes a pathway may be endogenous to the host cell, but such is also not required, and a complete pathway or components of a pathway may be introduced into a host cell.

Cell Free System.

“Cell-free system,” as used herein, is an isolated cell-free system containing a cell lysate or extract expressly engineered to synthesize an enzyme or cascade of enzymes that, when acting in a given sequence (e.g., in an enzymatic pathway) and proportion over a determined substrate, results in the preferential generation of a compound of interest. A compound of interest is typically a chemical entity (e.g., a small molecule), which can be used as an active pharmaceutical ingredient (API), chemical precursor, or intermediate.

“Substrate,” as used herein, is a compound or mixture of compounds capable of providing the required elements needed to synthesize a compound of interest.

“Adenosine triphosphate regeneration system” or “ATP regeneration system,” as used herein is a chemical or biochemical system that regenerates adenosine, AMP and ADP into ATP. Examples of ATP regeneration systems include those involving glucose metabolism, glutamate metabolism, and photosynthesis.

“Reducing equivalent,” as used herein, is a chemical species which transfers the equivalent of one electron in a redox reaction. Examples of reducing equivalents are a lone electron (for example in reactions involving metal ions), a hydrogen atom (consisting of a proton and an electron), and a hydride ion (:H—) which carries two electrons (for example in reactions involving NAD). A “reducing equivalent acceptor” is a chemical species that accepts the equivalent of one electron in a redox reaction.

Metabolite.

A metabolite is any substance used or produced during metabolism. For the purposes of the present invention, a metabolite is often, although not always, the product of an enzyme in the pathway of interest.

Inducible Expression.

The methods of the invention may make use of regulated expression of various coding sequences. Expression may be regulated by various cues, for example induction by chemicals, change of growth phase, depletion of a nutrient, temperature shifts, and/or light. In some embodiments inducible promoters regulated by the presence of an inducing agent, e.g. a chemical such as lactose, arabinose, or tetracycline, as known in the art.

Expression and cloning vectors usually contain a promoter that is recognized by the host organism and is operably linked to the coding sequence of interest. Promoters are untranslated sequences located upstream (5′) to the start codon of a structural gene that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. While the native promoter may be used, for most purposes heterologous promoters are preferred, as they generally permit greater transcription and higher yields.

Promoters suitable for use with prokaryotic hosts include the -lactamase and lactose promoter systems, alkaline phosphatase, a tryptophan (trp) promoter system, and numerous hybrid promoters such as the tac promoter. However, other known bacterial promoters are also suitable, e.g. the lacI promoter, the T3 promoter, the T7 promoter, the arabinose promoter, the gpt promoter, the lambda PR promoter, the lambda PL promoter, promoters from operons encoding glycolytic enzymes such as 3-phosphoglycerate kinase (PGK), and the acid phosphatase promoter. Their nucleotide sequences have been published, thereby enabling a skilled worker operably to ligate them to a sequence of interest using linkers or adaptors. Promoters for use in bacterial systems also will contain a Shine-Dalgarno (S.D.) sequence operably linked to the coding sequence. In certain cases, also, the host cell may be modified genetically to adjust concentrations of metabolite or inducer transporter proteins so that all cells in a culture will be induced equivalently.

### Production Methods

High yield production of a product of interest is accomplished by providing a cell in which cytoplasmic enzymes comprising a pathway of interest are expressed, e.g. at physiologically normal levels, or at greater than physiologically normal levels. For production purposes, a lysate of the cell is utilized. Cells are lysed by any convenient method that substantially maintains enzyme activity, e.g. sonication, French press, and the like as known in the art. The lysate may be fractionated and/or particulate matter spun out, or may be used in the absence of additional processing steps. The cell lysate may be further combined with substrates, co-factors and such salts, and/or buffers, as are required for enzyme activity.

Lysates of cells of different genetic backgrounds, e.g. previously altered or genetically engineered, or species, or that are prepared by different strategies can be mixed and simultaneously or sequentially used in a bioprocess with the cell lysate of the invention. The lysate can be free or immobilized or can be sequestered in the reactor by ultrafiltration or other means while removing the product, and can be reused or disposed at each stage of the process.

The methods of the invention provide for high yields of the desired product, which yield is greater than the yield that can be achieved with a native microbial host. Productivity (i.e. rate of production per unit of volume or biomass) may also be increased. In one embodiment of the invention, the yield of product is at least about five-fold the basal rate, at least about 10-fold the basal rate, at least about 25-fold the basal rate, or more. The methods may also increase the efficiency of converting the substrate into the product where the conversion efficiency may be increased by 5%, 10%, 20% or more relative to the basal conversion efficiency of the native microbial host.

Different inocula can be adapted to different conditions (e.g. two batches grown on two different carbon sources) or can have different genotypes and then mixed to carry out the process (e.g. to get simultaneous consumption of a mix of carbon sources or sequential processing of a metabolite through a pathway divided in two separate batches of cells). A process can also take place sequentially by allowing one set of reactions to proceed in one vessel and then passing the supernatant or filtrate through a second vessel.

The reactions may utilize a large scale reactor, small scale, or may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced over time to prolong the period of time for active synthesis. A reactor may be run in any mode such as batch, extended batch, semi-batch, semi-continuous, fed-batch and continuous, and which will be selected in accordance with the application purpose.

The reactions may be of any volume, either in a small scale, usually at least about 1 ml and not more than about 15 ml, or in a scaled up reaction, where the reaction volume is at least about 15 ml, usually at least about 50 ml, more usually at least about 100 ml, and may be 500 ml, 1000 ml, or greater up to many thousands of liters of volume. Reactions may be conducted at any scale.

Various salts and buffers may be included, where ionic species are typically optimized with regard to product production. When changing the concentration of a particular component of the reaction medium, that of another component may be changed accordingly. Also, the concentration levels of components in the reactor may be varied over time.

In a semi-continuous operation mode, the reactor may be operated in dialysis, diafiltration batch or fed-batch mode. A feed solution may be supplied to the reactor through the same membrane or a separate injection unit. Synthesized product is accumulated in the reactor, and then is isolated and purified according to the usual method for purification after completion of the system operation. Alternatively, product can be removed during the process either in a continuous or discontinuous mode with the option of returning part of or all of the remaining compounds to the reactor.

Where there is a flow of reagents, the direction of liquid flow can be perpendicular and/or tangential to a membrane. Tangential flow is effective for preventing membrane plugging and may be superimposed on perpendicular flow. Flow perpendicular to the membrane may be caused or effected by a positive pressure pump or a vacuum suction pump or by applying transmembrane pressure using other methods known in the art. The solution in contact with the outside surface of the membrane may be cyclically changed, and may be in a steady tangential flow with respect to the membrane. The reactor may be stirred internally or externally by proper agitation means.

The amount of product produced in a reaction can be measured in various fashions; for example, by enzymatic assays which produce a colored or fluorometric product or by HPLC methods. One method relies on the availability of an assay which measures the activity of the particular product being produced.

During the biosynthetic process, the cell-free system is monitored for the concentration of metabolic parameters, as described herein. When the concentration of a metabolic parameter varies by a predetermined level from the target range, i.e. a target concentration determined to provide for optimized biosynthesis of the desired pathway product; the system is adjusted to bring the concentration of the metabolic parameter back to a desired target range.

When reducing equivalents are required for function of the biosynthetic pathway, various methods may be utilized to increase the availability of NADH. In some embodiments a source of reducing equivalents is channeled into an enzymatic pathway that reduces NADP to NADPH. For example, glucose can be preferentially channeled to the pentose phosphate shunt by augmenting the reaction mix with glucose-6-phosphate dehydrogenase and/or 6-phosphogluconolactonase. In combination with augmentation, or as an alternative, the reaction mix can be treated with a protease to inactivate an enzyme in the standard glycolytic pathway allowing preferential flux of glucose to the pentose phosphate shunt. Alternatively such reducing equivalents are obtained from aliphatic substrates; by augmenting the reaction mixture with enzymes transferring reducing equivalents from these substrates to NADP; and the like.

When biosynthetic reactions of interest produce excess reducing equivalents, various methods may be utilized to remove the excess electrons and recycle NADP+ or NAD+. In some embodiments, an active electron transport chain is provided, e.g. by including vesicles active in oxidative phosphorylation, where O₂ is present as an electron receptor. In such reactions, O2 is metered into the biosynthesis reaction at a rate sufficient to produce the desired balance of NADP+ and/or NAD+. For the desired redox flux it may be necessary to uncouple ATP formation from the rate of electron delivery to O₂. Methods of uncoupling include addition of uncoupling compounds, e.g. dinitrophenyl; addition of a pyridine nucleotide transhydrogenase enzyme to transfer reducing equivalents from NADPH to NAD+. It may also be necessary to transfer electrons between NADPH and NADH using transhydrogenases or other means.

As described herein, various adjustments to central metabolism can be pursued to achieve the desired adjustment in parameter concentration. Generally, redox flows between NAD(H) and NADP(H) can be adjusted with modulation of the activity of transhydrogenase, to transfer reducing equivalents from NADPH to NADH. A need for reducing equivalents for biosynthesis can be adjusted with modulation of energy from glycolysis to the pentose pathway, e.g. by increasing activity of glucose-6-dehydrogenase. More energy can be diverted to glycolysis by increasing O₂ and glucose phosphate isomerase.

Conveniently an automated system is provided, in which monitoring and adjustments are performed automatically.

### EXEMPLIFICATION

The following examples are put forth so as to provide those of ordinary skill in the art with a complete disclosure and description of how to make and use the present invention, and are not intended to limit the scope of the invention or to represent that the experiments below are all or the only experiments performed. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Development of a Cell Free Metabolic Control Test Rig

The development of cell-free metabolic systems provides a potential for direct on-line control of a metabolic reaction network. The absence of the cell wall and dispersion of the macromolecular catalysts throughout the entire reaction volume allows precise sampling for on-line monitoring as well as immediate dispersion of added substrates and reaction control reagents. Complex biological conversions can be approached using technologies employed by traditional heterogeneous and homogeneous catalysis processes. Such bioconversion processes can take advantage of the decades of development that has been effective for the large-scale production of commodity chemicals.

However, in order to achieve an ultralow cost target, synthesis will be performed with use of crude cell lysates, and even when the enzymes for targeted biosynthetic pathways have been overexpressed, such lysates contain hundreds of different catalysts. Further, much of the central metabolic network must be maintained in order to provide pathway precursors (substrates), to either provide or to remove reducing equivalents, and to direct chemical energy (ATP and GTP) as required for efficient product formation. Just as for processes using chemical catalysis, monitoring methods and system perturbation experiments can be used to determine response time constants and the degree of subsystem connectivities in order to determine the nature of the control actions that will obtain the most effective process performance.

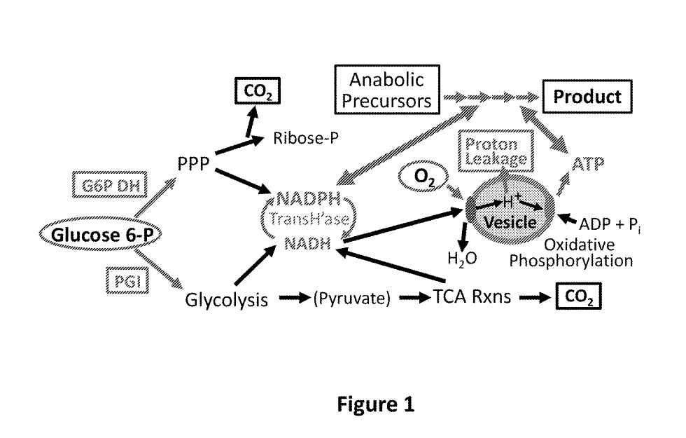

FIG. 1 provides a simplified diagram that shows foundational concepts in metabolism. It assumes that glucose is the principle carbon and energy source, that the glucose is continually added at a controlled rate, and that it is quickly phosphorylated by glucokinase using ATP as the phosphate source. Compounds shown as surrounded by blue ovals are fed into the reactor as needed to control metabolism. Blue rectangular boxes and blue arrows represent biochemical processes whose rates are adjusted. G6P DH represents glucose 6-P dehydrogenase, the enzyme that takes glucose 6-P into the pentose phosphate pathway (PPP), and PGI is phosphoglucose isomerase, the enzyme that controls the flux of glucose toward glycolysis and the TCA cycle. The relative activities of these enzymes determine the relative rates of NADH vs. NADPH formation as reducing equivalent carriers. Also, the transhydrogenase (TransH'ase; or a similar activity) is used to transfer reducing equivalents from NADPH to NADH as required.

The system is controlled through altering enzyme activity, O₂, and proton leakage to achieve the desired regulation of cell-free metabolic reactions. For example, if an anabolic pathway requires many reducing equivalents, more of the glucose is shunted through the PPP pathway, for example by increasing activity of glucose-6-P dehydrogenase.

Alternatively, if a pathway requires high levels of ATP and few reducing equivalents, more of the glucose can be shunted to glycolysis and the TCA cycle, by increasing O₂ concentration and PGI activity. The PPP vs. glycolysis balance must also reflect which anabolic precursors are required. If these are all pyruvate or pyruvate derivatives, then enough glucose must go through glycolysis to satisfy this need. If, on the other hand ribose phosphate is an important precursor, the PPP must supply this.

Consideration is also given to metabolic pathways that produce excess reducing equivalents but require only small amounts of ATP. In this case, the reducing equivalents must be accepted by oxygen without producing ATP. However, the transfer of the reducing equivalents to oxygen will create a proton gradient across the membrane of the vesicle. If this proton gradient is not relieved by ATP generation, the proton motive force will accumulate to slow down or even stop the acceptance of electrons. In this case, an agent is added, for example, dinitrophenol, that allows the protons to leak across the membrane to relieve this gradient and allow more electron flux to oxygen.

The rate of oxygen addition is controlled to help balance the metabolic system to ensure that enough reducing equivalents and ATP are available for the biosynthetic pathway. Examples are shown in Table 1.

TABLE 1

| | Requirements | | | | Actions | | | | |
|---|---|---|---|---|---|---|---|---|---|
| | | | | Need | Increase | | | | |
| | Need | Need | Need | Pyruvate | G6P | Increase | Increase | Increase | Leak |
| CASE | NADPH | Ribos-P | ATP | Derivative | DH | PGI | TransHase | O₂ | Protons |
| 1 | Yes | Yes | No | No | Yes | No (-) | No (-) | No | No |
| 2 | No | Yes | No | No | Yes | No (-) | Yes | Yes | Yes |
| 3 | No | No | No | Yes | No | Yes | No (-) | Yes | Yes |
| 4 | No | No | Yes | Yes | No | Yes | No (-) | Yes | No |

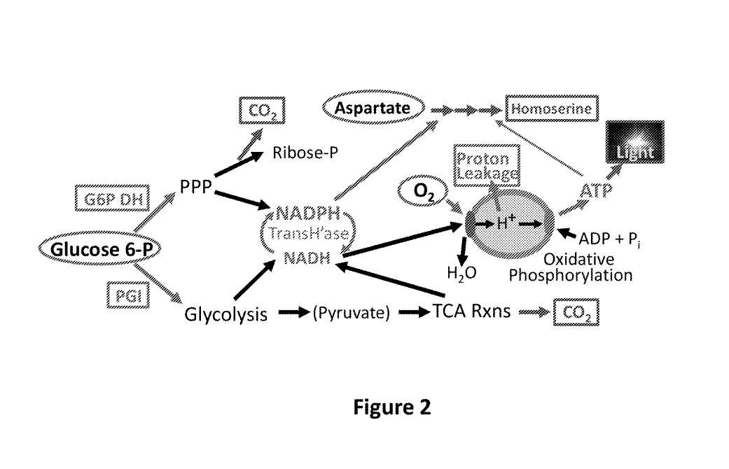

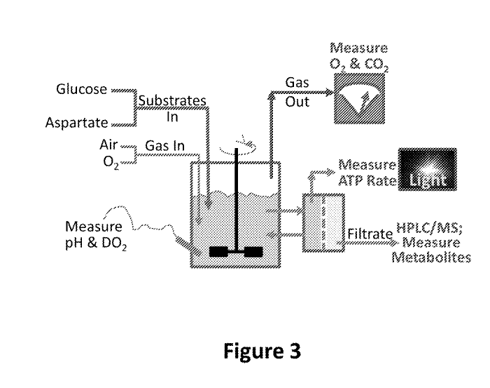

In order to evaluate control response capabilities and dynamics for a simple biosynthetic pathway, a test rig may be constructed. For example, a pathway can be chosen that requires both reducing equivalents (NADPH) and chemical energy (ATP). The conversion of aspartic acid to homoserine uses three consecutive enzymes and requires two NADPH reducing equivalents and one ATP, shown in FIG. 2. The factors that are manipulated are shown in blue and the response parameters are shown in magenta. The actual test rig is diagrammed in FIG. 3. The feed rates of glucose and aspartic acid are separately adjusted, as are the addition rates for air and oxygen. The concentrations of G6PDH, PGI, and the amount of dinitrophenol are independently manipulated both for basal metabolism determinations and for determining responses to step changes in each of these parameters. The cell-free metabolic reactor is operated in continuous mode to simulate efficient large scale operation in which the catalysts (enzymes) are retained by an ultrafiltration membrane and the filtrate is removed at the same rate as the substrates are fed. The dissolved oxygen concentration and pH are continuously monitored and controlled. Oxygen consumption and CO₂ evolution are determined by measuring the gas concentration in the exit gas stream and conducting a material balance. Metabolite concentrations as well as NADP and NADPH concentrations are frequently determined using a rapid turnaround HPLC/MS instrument. Finally, the rate of ATP production is determined by taking a side stream of the reactor contents into a flow cell where luciferase and luciferin are added and the resultant luminescence intensity measured.

What is claimed is:

1. A method of controlling metabolic flux rate in a cell-free system comprising a complex set of enzymes, to produce a desired product of a pathway of interest, the method comprising:
taking measurements of metabolic performance;
adjusting metabolic performance based on the measurements by performing one or more steps comprising: (i) altering enzyme levels in the cell-free system; (ii) altering feed rate of a substrate that controls redox flux or carbon flux to the cell-free system; (iii) altering O₂ addition to the cell-free system; (iv) controlling efficiency of electron transport system by altering leakage across a membrane;
wherein enzymes present in the pathway of interest catalyze production of the desired product.

2. The method of claim 1, wherein the measurement of metabolic performance comprises measurement of an adenine metabolite.

3. The method of claim 2, wherein the adenine metabolite is a nicotinamide adenine dinucleotide.

4. The method of claim 3, wherein the nicotinamide adenine dinucleotide is one or more of NAD, NADH, NADP and NADPH.

5. The method of claim 1, wherein the measurement of metabolic performance comprises measurement of ATP or ADP.

6. The method of claim 1 wherein dissolved oxygen concentration and pH are continuously monitored and controlled.

7. The method of claim 1, wherein the step of altering enzymes in the cell-free system comprises increasing activity of glucose-6 phosphate dehydrogenase.

8. The method of claim 1, wherein the step of altering enzymes in the cell-free system comprises increasing activity of phosphoglucose isomerase.

9. The method of claim 1, wherein the step of altering enzymes in the cell-free system comprises increasing transhydrogenase activity.

10. The method of claim 7, wherein the step of increasing activity comprises addition of the enzyme to the cell-free system.

11. The method of claim 7, wherein the step of increasing activity comprises addition of a coding sequence for said enzyme to the cell-free system, wherein the coding sequence is translated.

12. The method of claim 1, wherein O₂ is increased in response to said taking measurements.

13. The method of claim 1, wherein the step of altering leakage across a membrane comprises addition of dinitrophenol to said cell-free system.

14. The method of claim 1, wherein when the measurements indicate the metabolic performance would benefit from increased concentrations of NADPH and ribulose-5-phosphate, the enzyme activity of glucose-6 phosphate dehydrogenase is increased.

15. The method of claim 1, wherein when the measurements indicate the metabolic performance would benefit from increased concentrations of ribulose-5-phosphate without increased NADPH, the enzyme activity of glucose-6 phosphate dehydrogenase and transhydrogenase is increased; O₂ is increased; and proton leakage is increased.

16. The method of claim 1, wherein when the measurements indicate the metabolic performance would benefit from increased concentrations of pyruvate derivatives, the enzyme activity of glucose phosphate isomerase is increased; O₂ is increased; and proton leakage is increased.

17. The method of claim 1, wherein when the measurements indicate the metabolic performance would benefit from increased concentrations of ATP and pyruvate derivatives, the enzyme activity of glucose phosphate isomerase is increased; and O₂ is increased.

18. The method according to claim 1, wherein the cell-free system comprises a microbial cell lysate.

19. The method of claim 18, wherein the microbial cell lysate is utilized without fractionation.
